(19) United States
(12) Patent Application Publication
(10) Pub. No.: US 2026/0076764 A1
(43) Pub. Date: Mar. 19, 2026

(54) MEDICAL MICROSCOPE AND MICROSCOPE SYSTEM

(71) Applicant: Jadent GmbH, Aalen (DE)

(72) Inventors: Klaus-Juergen JANIK, Neuler (DE); Ulrich WOESSNER, Geislingen (DE)

(73) Assignee: Jadent GmbH, Aalen (DE)

(21) Appl. No.: 19/327,655

(22) Filed: Sep. 12, 2025

Related U.S. Application Data
(63) Continuation of application No. PCT/EP2024/056826, filed on Mar. 14, 2024.

(30) Foreign Application Priority Data
Mar. 15, 2023 (DE) 10 2023 106 506.8

(51) Int. Cl. A61B 90/25; G02B 21/20

(52) U.S. Cl. CPC A61B 90/25; G02B 21/20

(57) ABSTRACT

A medical microscope includes an eyepiece system having a binocular establishing a viewing direction into the microscope. A microscope body has an objective lens for capturing light from a field of view and directs the light to the eyepiece system and has a microscope axis extending from the microscope body to the field of view. A suspension secures the microscope to a support system and is designed as part of a ball-and-socket joint system. When the suspension is in a non-locked state, it allows the medical microscope to rotate about a reference point of the suspension. The suspension is positioned with respect to a center of gravity of the microscope such that the microscope body is oriented such that the microscope axis extends in a predefined direction and the viewing direction extends at a predefined angle range relative to a suspension axis extending through the center of gravity and suspension.

### CROSS-REFERENCE TO RELATED APPLICATIONS

This application is a continuation under 35 U.S.C. § 120 of International Patent Application No. PCT/EP2024/056826, filed Mar. 14, 2024, which claims the benefit of German Patent Application No. 10 2023 106 506.8, filed Mar. 15, 2023, the contents of each of which are incorporated by reference herein.

The present invention relates to a medical microscope with a microscope hanging mount, in particular for flexibly positioning a microscope. The invention also relates to a microscope system.

In medical applications, microscopes are used for the examination and/or treatment of patients. They are referred to as medical microscopes, surgical (operation OP) microscope, diagnostic microscope, examination microscope, or simply microscope. Medical microscopes are used in particular in microsurgery, for example, in neurosurgery (e.g., surgery in the area of the head and intervertebral discs), ophthalmology (e.g., cataract surgery), plastic surgery (e.g., cosmetic surgery) or dental medicine (e.g., root canal treatment, implantology, . . . ). Medical microscopes are typically used for stereoscopically captured views of tissue to be examined or treated and provide significant, often adjustable, magnification factors.

A medical microscope comprises a binocular observation (e.g., Keplerian tube) in conjunction with, e.g., a multi-stage switchable magnification changer according to Galileo (telescope system) in combination with coaxial illumination (e.g., with a 6 V/30 W incandescent lamp), which is fed into the microscope beam path coaxially via a mirror, for example. This basic design corresponds to the first surgical microscopes from 1953 by Dr. Littmann (Zeiss AG) and Prof. Wullstein (University of Würzburg), with which the first microsurgical operations in the ENT field were successfully performed. This was followed by the use of microscopes in ophthalmology, neurosurgery, gynecology, urology, and, at the end of the 1990s, in dental medicine.

Using a detachable coupling system, medical microscopes are held in place by a flexible support system, such as a ceiling, wall, or floor stand, or by a fixed support system.

The coupling system usually comprises a hanging mount mechanics that detachably connects the microscope to the support system, for example, microscope mounting arm, and optionally can provide one or more degrees of freedom. Parts of the coupling system (herein also referred to as microscope hanging mount) are formed on the microscope and on the support system. One objective of the support system and the microscope hanging mount is to enable free positioning of the microscope in relation to the patient being examined and to provide the most rigid (fixed) positioning of the microscope possible during the examination/treatment so that a capturing of the field of view with the microscope can be performed that is as free from shake as possible.

The state of the art includes a wide variety of configurations of microscopes and microscope hanging mounts. Optical configurations of microscopes relate, in particular, to the configuration of a microscope body and an ocular system. The microscope body comprises usually an objective system comprising, for example, a magnification system (e.g., a magnification changer such as a Galilean changer) and a main objective with one (or more) patient-side objective or main lens of the microscope. Depending on the application, magnification systems can provide several, for example 3-6, magnification levels or a ZOOM system with continuous magnification adjustment. The main objective can be configured, for example, as a vario-objective with adjustable focal lengths. The ocular system comprises one or more ocular lenses with the respective tubes for each of the two partial beam paths that also are referred to as observation tube. Typical magnifications of microscopes with binocular systems range from 2.5× to 30× or more.

There are various microscope hanging mounts that, e.g., are adapted to the various types of stands used in the respective medical environment. Hanging mount mechanics are common that engage on one side of the microscope and, thus, extend aside the microscope and that provide a degree of freedom of movement in the form of a forward and backward pivot movement of the microscope for positioning the field of view on the patient.

If the objective system and ocular system of a microscope are rigidly connected to each other, the operator must follow the movement of the microscope with his head when it is moved. Manufacturers of such microscopes include, in addition to the applicant Jadent GmbH, inter alia Zeiss AG, Leica Microsystems GmbH, Karl Kaps GmbH & Co. KG, and Global Surgical Corporation.

The inventors have recognized disadvantages of known microscopes hanging mounts, in particular of lateral hanging mounts, in the medical use, such as spatial restrictions when positioning a microscope and a presence of spatial limitations, e.g., for the surgery assistance. Thus, it was generally recognized that there is a need for microscopes, particularly low-cost microscopes, that have a field of view that can be positioned as easily as possible in preparation for or during treatment/surgery.

An aspect of this disclosure is therefore based on the objective of providing a compact and cost-effective setup of a microscope system that allows flexible setting of the position of the microscope and, thus, the field of view—preferably in three-dimensional space. A further aspect of this disclosure is based on the objective that the microscope should restrict the operator and the supporting personal as less as possible and/or that the exchange with other people during the examination/treatment is not hindered as far as possible.

At least one of these objectives is solved by a medical microscope according to claim 1 and by a microscope system according to claim 10. Further developments are given in the dependent claims.

In an aspect, a medical microscope comprises a microscope ocular system with a binocular, wherein the binocular provides an observation direction into the microscope, and a microscope body with at least one objective lens for capturing light from a field of view, wherein the microscope body is configured to transmit the light to the micro-scope ocular system and has a microscope axis extending from the microscope body to the field of view. Moreover, the medical microscope comprises a microscope hanging mount for mounting the microscope to a support system, wherein the microscope hanging mount is configured as part of a ball joint system and, in a non-locked state of the microscope hanging mount, provides rotatability of the medical microscope about a reference point of the microscope hanging mount. The microscope hanging mount is positioned relative to a center of gravity of the micro-scope in such a way that, when the microscope is attached to the support system in a non-locked state of the microscope hanging mount, the microscope body assumes a basic orientation in which the microscope axis extends in a predetermined use-observation direction in three-dimensional space and the observation direction extends at a predetermined use-angle range with respect to a hanging mount axis extending through the center of gravity and the microscope hanging mount.

In an aspect, a microscope system comprises a support system, which is configured in particular as a floor, wall or ceiling stand or as a permanently mounted support system, and

a medical microscope as described above, which is mounted to the support system with a microscope hanging mount of a microscope ocular system of the medical microscope. The microscope hanging mount provides, in particular in a non-locked state, multi-axis freedom of movement, so that the mounted medical microscope assumes a basic orientation in the non-locked state, in which a microscope axis of the medical microscope (9) extends in a predetermined use-observation direction in three-dimensional space, in particular in a vertical direction downwards or at an angle of 0° to 5° to the vertical direction, and an observation direction extends at a predetermined use-angle in the range of 0° to 90°, in particular in the range from 25° to 70°, to a hanging mount axis extending through the center of gravity and the microscope hanging mount.

In some embodiments of the medical microscope, the microscope axis can extend parallel offset or coaxially to the hanging mount axis or it can extend at an angle in a range from 0° to 5° to the hanging mount axis or in a range from 20° to 90° with respect to the hanging mount axis. Alternatively or additionally, the use-angle range can comprise angles in the range from 0° to 90°, in particular in the range from 25° to 70°, with respect to the hanging mount axis.

In some embodiments, the medical microscope can further comprise a center of gravity translation unit, which is arranged between the microscope hanging mount and the microscope body, in particular between the microscope hanging mount and an ocular base body of the microscope ocular system, and is configured for translating the microscope body in one or two directions, in particular for changing a position of the center of gravity relative to the microscope hanging mount. Furthermore, the center of gravity translation unit can be configured to position the center of gravity of the medical microscope with regard to adjusting the basic orientation to at least two configurations of the medical microscope, which differ in the position of the center of gravity, so that the basic orientation in the at least two configurations of the medical microscope is settable in accordance with the predetermined use-observation direction in three-dimensional space when the microscope hanging mount is in the non-locked state.

In some embodiments, the medical microscope can be provided with at least one degree of freedom of movement, which provides an orientability of the microscope axis with respect to the microscope ocular system, and the basic orientation is present in a basic setting of the medical microscope with respect to the degree of freedom of movement, wherein the basic setting provides a deflection in two opposite directions enabled by the degree of freedom. Furthermore, the hanging mount axis of the medical microscope can be defined in the basic setting of the medical microscope.

In some embodiments, the microscope hanging mount can be arranged on the microscope ocular system and/or on the microscope body.

In some embodiments, the medical microscope can further comprise an opto-mechanical system arranged between the microscope ocular system and the microscope body and feeding light captured with the microscope body to the binocular. The opto-mechanical system can be configured for a pivot movement of the microscope body about a pivot axis and can comprise a first rotation unit for a rotational movement of the microscope body about a first rotation axis given by the first rotation unit, wherein the first rotation axis extends coaxially, parallel or at an angle in the range from 0° to 5° to a section of the optical axis of symmetry that extends through the first rotation unit.

In some embodiments of the microscope system, the support system can comprise a microscope mounting arm, which engages the microscope hanging mount of the microscope ocular system at an angle in the range from 0° to 20° with respect to a vertical direction, in particular vertically from above. Alternatively or additionally, the support system can comprise a pivot joint that enables rotatability of the attached microscope, when the microscope hanging mount is locked, wherein a rotation axis of the pivot joint extends in particular, coaxially or parallel offset to the use-observation direction, for example, vertically.

In some embodiments of the microscope system, the support system can engage the microscope hanging mount of the microscope ocular system at an angle in the range from 0° to 90° with respect to a vertical direction, in particular, obliquely from above.

Disclosed herein are concepts that allow at least partial improvement of aspects from the prior art. In particular, further features and their usefulness will result from the following description of embodiments based on the figures. From the figures show:

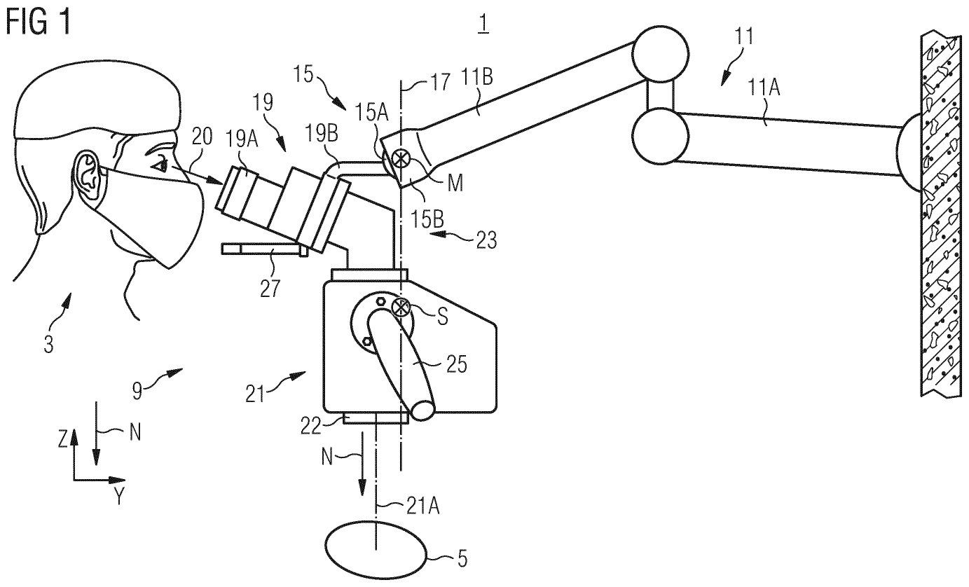

FIG. 1 a schematic representation of a microscope system for illustrating the inventive concept,

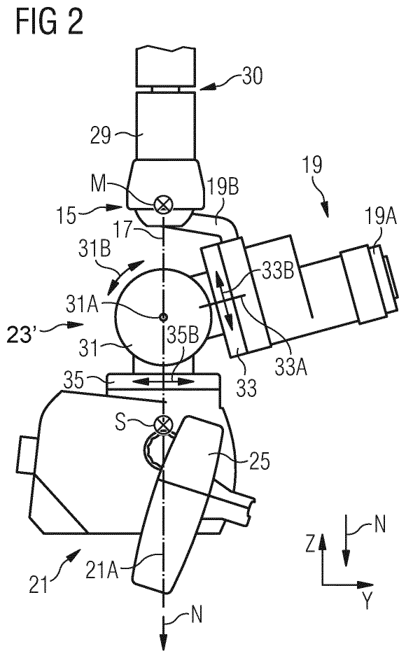

FIG. 2 a schematic of an implementation of the inventive concept in a further configuration of a medical microscope,

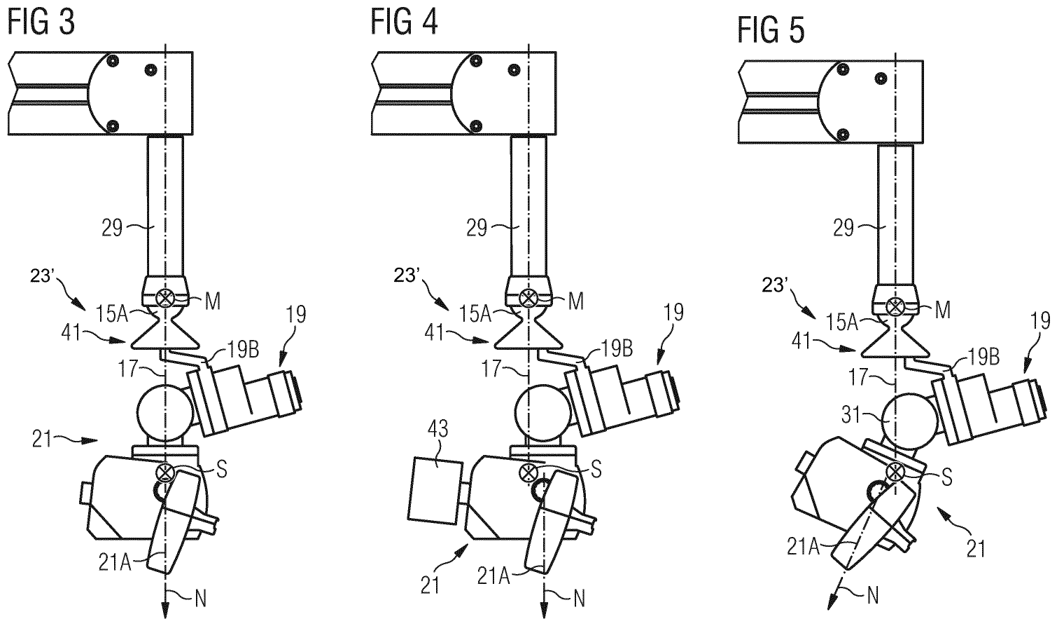

FIG. 3 to FIG. 5 schematic representations of a medical microscope with different configurations and with adapted location of the center of gravity, and

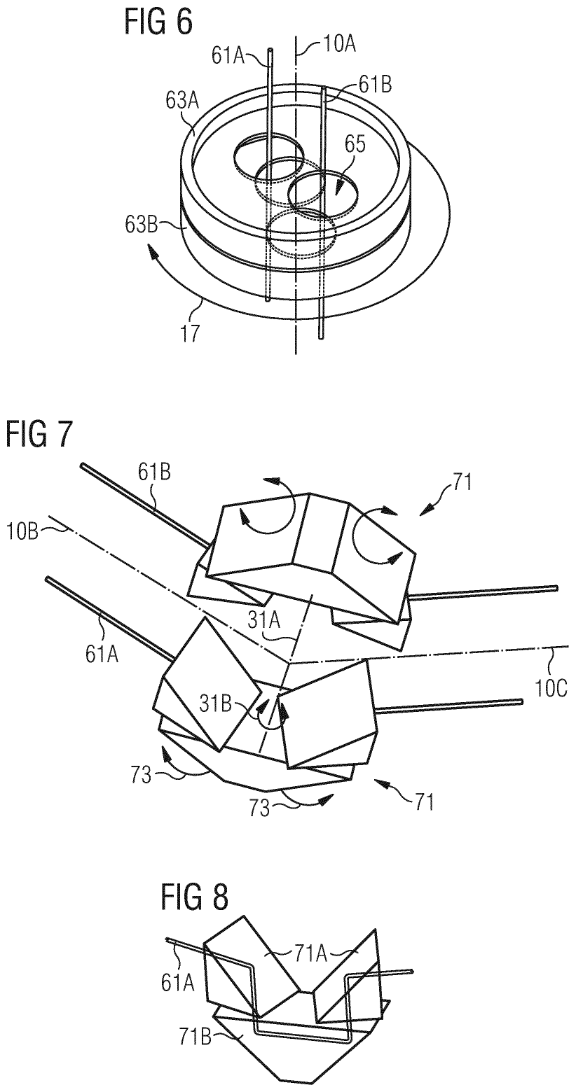

FIGS. 6 to 8 schematic representations for illustrating an exemplary rotation unit and an exemplary pivot unit.

The concepts proposed by the inventors enable shifts of the microscope with non-locked microscope hanging mount, essentially without the microscope changing its position in space on its own (movements of the microscope could occur due to the force of gravity).

FIG. 1 shows in a schematic illustration a microscope system 1, which allows an operator 3 to view a field of view 5 (indicated as circular in FIG. 1) to be examined with a medical microscope 9 under magnification. The microscope 9 is held by a support system 11. The support system 11 comprises, for example, a (ceiling, wall, floor) stand with a rear arm 11A and a spring arm 11B. The stand is configured in such a way that the microscope 9 can be (roughly) positioned as freely as possible in space in terms of height (Z-direction) as well as in the horizontal plane (X-and Y-direction, herein the plane in which the field of view 5 extends) with respect to a patient to be examined/treated (gross motor movements).

A use-observation direction N toward the patient to be examined/treated extends from the microscope 9 to the field of view 5.

The microscope hanging mount of the microscope 9 is configured as part of a ball joint system 15 (i.e., as a multi-axis joint for free adjustment around degrees of freedom with respect to several (three) axes). In FIG. 1, the microscope hanging mount is a ball 15A of the ball joint system 15. Together with a second part of the ball joint system 15 (here a ball guide 15B on the spring arm 11B), the microscope hanging mount generally forms a hanging mount device with several degrees of freedom of movement. The free movability of the ball in the ball guide corresponds to free movement around three orthogonal axes of rotation. The hanging mount device is intended for the detachable mounting of the microscope 9 to the support system 11, which, in a non-locked state, enables degrees of freedom of movement and, in a locked state, provides a fixed mounting of the microscope 9 to the support system 11. Alternatively, an inverted arrangement of the ball joint system 15 with a ball on the spring arm 15 can be provided.

In general, the ball joint system 15 and, optionally, the mechanics of the support system 11 can be locked (e.g., mechanically or magnetically). This allows that a further movement of the microscope 9 is prevented once a desired position of the microscope 9 has been reached.

In the non-locked state of the ball joint system 15, the microscope 9 will orient itself in space with a center of gravity S of the microscope 9 below a reference point of the ball joint system 15 (center point M of the ball). This self-determined (balanced) position of the microscope 9 in three-dimensional space for a non-locked state of the ball joint system 15 is referred to herein as the basic orientation of the microscope 9. In the basic orientation, the center of gravity S and the center point M define a hanging mount axis 17. The ball joint system 15 enables the basic orientation to be taken independently of the support system 11, i.e., for example, both in the case of an inclined microscope mounting, e.g., at an angle of 60° with respect to the horizontal plane, and in the case of a vertically mounted microscope mounting. FIG. 1 shows the microscope 9 in its basic orientation for an inclined microscope mounting; FIGS. 2 to 5 show microscopes in the basic orientation for a vertically applied microscope mounting (vertical mounting).

In FIG. 1, the microscope 9 also comprises an ocular system 19 with a binocular 19A and a base body 19B, whereby the ball 15A of the ball joint system 15 is also provided on the base body 19B. Thereby, an observation direction 20 into the binocular 19A is provided, for example, with respect to the reference point of the ball joint system 15 and, thus, the hanging mount axis 17.

The microscope 9 further comprises a microscope body 21 with at least one objective lens 22 for capturing light from the field of view 5. FIG. 1 shows a microscope axis 21A extending from the microscope body 21 to the field of view 5 (in particular, from the center of the objective lens 22 to the center of the field of view 5).

The microscope body 21 is mounted to the base body 19B via a schematically represented optical unit 23. The microscope body 21 and the optical unit 23 are configured to transmit the captured light to the microscope ocular system 19.

If the microscope body 21 is rigidly connected to the microscope hanging mount, for example, via the optical unit 23, the microscope axis 21A is spatially set with respect to the reference point of the ball joint system 15.

In addition, a settability of the microscope body 21 can be given as, for example, an opto-mechanical unit 23′with at least one degree of freedom of movement connects the microscope body 21 to the microscope hanging mount (see FIG. 2). In this case, the microscope 9 can be assigned a basic setting in which the microscope axis 21A is spatially fixed relative to the reference point of the ball joint system 15.

If there is, in general, an orientability of the microscope axis 21A with respect to the microscope ocular system 19 and the microscope hanging mount in one degree of freedom of movement, the basic orientation is usually present in the basic setting of the medical microscope 9 with regard to the degree of freedom of movement in such a way that the basic setting provides a displacement in two opposite directions made possible by the one degree of freedom of movement. Usually, a central orientation of the microscope axis 21A with regard to the degree of freedom of movement is provided as the basic setting. If, for example, the basic setting is in a middle range of a degree of freedom of movement provided by the opto-mechanical system 23′, the operator can be provided with a sufficient degree of movement in both directions of the degree of freedom based on the basic setting. In the basic setting of the microscope 9, there may also be provided a desired observation direction into the binocular 19A.

For positioning the microscope 9 and, thus, the field of view 5, hand gripes 25 are usually attached to the microscope body 21, for example on the side of the microscope body 21. FIG. 1 also shows a mouth switch 27, which can be operated by the operator 3, for example, to control a drive of the microscope 9, in particular, the microscope body 21, and/or a locking device of the ball joint system 15 or of the support system 11 and/or an autofocus function.

The use-observation direction N is given by the microscope axis 21A assigned to the microscope 9, in particular, in the basic setting for a spatially settable microscope body. In the use-observation direction N, medical microscopes provide different working distances between the microscope body and the field of view, depending on the field of application. For example, the working distance of a focal plane of the microscope perpendicular to the microscope axis 21A, in which the field of view 5 is located, to the objective lens 22 of the microscope body 21 is in the range of f=175-200 mm in ophthalmology or in the range of f=300-420 mm in neurosurgery.

As already mentioned, FIG. 2 shows an embodiment of a vertical mounting with the microscope 9 attached to a microscope mounting arm 29, which extends vertically below the spring arm 11B; in general, the microscope mounting arm 29 can extend at an angle in the range from 0° (from above) to 20° with respect to a vertical direction.

In addition to the ball joint system 15 arranged at the end of the microscope mounting arm 29, a further degree of rotational freedom is provided in the microscope mounting arm 29 with a (in particular, lockable) rotary joint 30. The rotary joint 30 allows the mounted microscope 9 to be rotated about a rotational axis when the ball joint 15 is locked. The rotational axis extends in a vertical (Z) direction, e.g., like the linear extension of the microscope mounting arm 29. While maintaining the position of the focal plane and the use-observation direction N, and with the observation direction into the binocular 19A unchanged with respect to the vertical, the orientation of the operator 3 in the X-Y plane relative to the patient to be examined/treated can, thus, be varied. An example application of the associated orientation of the field of view to a geometry to be captured is the keratoplasty in ophthalmology.

In the embodiments shown in the figures as examples, the binocular 19A is firmly connected to the support system 11 in the locked state via the base body 19B and the microscope hanging mount (here the ball joint system 15); a fixed position of the binocular 19A relative to the support system 11 (and, thus, to the patient) is given. The observation direction can be inclined downward at an angle to the horizontal plane, e.g., in the range of 10° to 45° or in the range of −10° to 50° or ±10°. For example, a fixed orientation of the observation direction 20 into the binocular, for example at 20° or 45° to the horizontal plane, can be provided. Alternatively, an adjustable angle relative to the horizontal plane can be provided for orienting the observation direction, for example, with a binocular that can be set via an ocular pivot tube.

In the embodiments shown in FIGS. 2 to 5, the microscope body 21 is attached to the base body 19B with the opto-mechanical system 23′. The—although limited—settability of the microscope body 21 relative to the ocular system 19 makes it possible to track the field of view 5 during treatment/surgery with the binocular 19A fixed in space (ball joint system 15 is locked). For this purpose, the microscope axis 21A is redirected, for example, from the vertical direction using the opto-mechanical system 23′.

FIG. 2 also shows the basic orientation of the microscope 9 in the basic setting with the opto-mechanical system 23′; i.e., there is a vertical hanging mount axis 17 and an orientation of the microscope axis 21A according to the use-observation direction N. Accordingly, the orientation of the microscope axis 21A is set relative to the binocular 19A (and, thus, to the observation direction into the binocular 19A) and to the microscope hanging mount. For example, in FIG. 2, for the mounted microscope 9, the microscope axis 21A extends vertically (in the Z direction)—in the basic orientation and basic setting shown-and the focal plane extends horizontally in the X-Y plane.

The opto-mechanical system 23′indicated in FIG. 2 decouples the location of the field of view of the microscope 9 from the position of the ocular system 19 in space. It is an aim to enable shifting the field of view to some limited extent in the focal plane by means of pivot and rotation (pivot) movements of the microscope body 21 while the ocular system 19 remains in a fixed position. The shifting of the field of view over the object to be examined/treated is carried out without the operator 29 having to change during the examination/treatment the posture that he assumed at the beginning of an examination/treatment with regard to the position of the ocular system 19 in space, which he has selected.

A pivot unit allows a pivot movement around a pivot axis (also referred to as tilting), whereby the pivot axis is transverse to a section of an optical axis of symmetry assigned to the pivot unit,. A rotation unit enables a rotational movement about a rotation axis, which is coaxial or parallel offset or essentially parallel, i.e., with an angular deviation of a few degrees, for example deviations in the range from 0° to 5°, to a section of the optical axis of symmetry associated with the rotation unit.

The shift in the field of view can be performed linearly, for example (illustrated in FIG. 2 based on a degree of freedom of a pivot unit 31). For clarification, FIG. 2 shows a pivot movement of the microscope body 21 around a pivot axis 31A indicated by an arrow 31B. The pivot movement is accompanied by a forward-backward displacement of the field of view, whereby exemplarly in FIG. 2 the pivot axis 31A can be aligned orthogonally and generally at an angle of 75° to 105° to the microscope axis 21A.

Furthermore, the shift of the field of view can be performed along a circular path (shown in FIG. 2 based on a degree of freedom of a first rotation unit 33, arrow 33B). The first rotation axis 33A can be oriented with the microscope axis 21A in a basic setting of the microscope 9 in an angle range of 25° to 90°. It can also be seen that, during a rotational movement about the first rotation axis 33A, an orientation of the first rotation axis 33A relative to the microscope axis 21A remains unchanged. In FIG. 2 one recognizes further that the pivot unit 31 is mounted to the ocular base body 19B by means of the first rotation unit 33, so that the first rotation unit 33 is configured for a rotational movement of the pivot unit 31—and, thus, of the microscope body 21 mounted to the pivot unit 31—about the first axis of rotation 33A. Accordingly, during a pivot movement about the pivot axis 31A, the orientation of the first axis of rotation 33A changes relative to the microscope axis 21A.

Based on a degree of freedom of a second rotation unit 35 (arrow 35B), the opto-mechanical system 23′ can introduce, as a further degree of freedom, a rotation of the microscope unit about an axis, for example, the optical axis of the microscope body 21. With this possibility of rotation of the microscope unit, the microscope unit can be rotated (e.g., for reasons of space or access) in the embodiment of the rotation unit described herein without significantly changing the captured image.

A combination of multidimensional displacement using the opto-mechanical system with an orientation of the field of view 5 using the hanging mount described herein with the ball joint system 15 can result in a degree of flexibility, ergonomics, and ease of use that is not known in the prior art.

For example, the orientation of the microscope axis 21A in space for an examination or operation can be further varied by the position of the microscope hanging mount. This can be done after positioning the microscope in the basic orientation and requires the corresponding orientation of the microscope 9. For this purpose, it must be possible to lock the microscope hanging mount in the corresponding position. If the binocular 19A is also equipped with degrees of freedom of movement, the orientation of the microscope axis 21A with respect to the binocular 19A is additionally determined by the orientation of the binocular 19A.

According to the invention, the microscope body 21 assumes in a non-locked state of the microscope hanging mount the basic orientation, in which the microscope axis 21A extends in a predetermined use-observation direction N in three-dimensional space and the observation direction 21B extends at a predetermined use-angle range with respect to the mounting axis 17 extending through the center of gravity S and the microscope hanging mount. The configuration of the binocular 19A and the (settable) microscope body 21 is, thus, selected such that a desired observation direction into the binocular 19A (if necessary, with a predetermined orientation of the binocular 19A) and a desired use-observation direction N are given.

In the embodiment shown in FIG. 1, for example, the microscope 9 has in the basic orientation its use-observation direction parallelly displaced to a hanging mount axis extending through the center of gravity S and the microscope hanging mount.

For the embodiment shown in FIG. 2, the basic setting is selected such that, when the ball joint 15 is not locked, the use-observation direction N of the microscope 9, the hanging mount axis 17, and optionally a rotation axis of the rotary joint 30 extend coaxially, for example, in FIG. 2, vertically in the Z direction.

The hanging mounts shown in FIGS. 1 and 2 are applied, as central hanging mounts, to the middle of the microscope 9. In particular, a central hanging mount can be configured as a vertical hanging mount extending vertically upwards, as shown in FIG. 2. Such hanging mounts enable that the view on the sides of the microscope body 9 is not restricted by protruding components of the hanging mount. Direct eye contact can contribute significantly to clearer communication and, thus, offers advantages in the course of the examination/treatment. A vertical hanging mount can further have the advantage that, in the basic setting of the microscope 9, the mechanical axis of the vertical hanging mount and the microscope axis 21A can extend displaced parallelly to each other (see, e.g., FIG. 1 or FIG. 3) or coaxially (see FIG. 2).

FIGS. 3 to 5 illustrate an embodiment of the microscope 9 in which additionally a center of gravity translation unit 41 is provided, which is arranged between the microscope hanging mount (ball 15A) and the microscope body 21. In particular, in the embodiment shown, the center of gravity translation unit 41 is arranged between the ball 15A and the ocular base body 19B of the microscope ocular system 19. The center of gravity translation unit 41 is configured for translation (relative translational movement between ball 15A and microscope body 21) in one or two directions, thus, enabling, in particular, the change of the position of the center of gravity S relative to the microscope hanging mount. In general, the center of gravity translation unit 41 is configured to position the center of gravity S of the medical microscope 9. The possibility of varying the position of the center of gravity results in an settability of the (balanced) basic orientation with respect to specific configurations of the microscope 9, as explained in more detail in connection with FIGS. 4 and 5 using two examples.

With the exception of the rotary joint 30 and the additionally provided center of gravity translation unit 41, the configuration shown in FIG. 3 corresponds to the embodiment schematically shown in FIG. 2 and described above.

The configuration shown in FIG. 4 differs from the configuration in FIG. 3 in that an additional optical component, for example, a camera 43, is provided on the microscope body 21, which leads to a different mass distribution attached to the support system 11 via the microscope mounting. Without interfering with the position of the center of gravity S with the center of gravity translation unit 41, the microscope 9 reorientates itself in such a way that the observation direction no longer extends in the desired use-observation direction N (in the Z direction).

If the center of gravity translation unit 41 displaces the microscope body 21 relative to the ball joint system (in FIG. 4, the base body 19B no longer engages centrally with the center of gravity translation unit 41), the desired orientation of the use-observation direction can be restored. In particular, it can be seen that in FIG. 4, the use-observation direction N extends slightly displaced parallelly to the hanging mount axis 17. Accordingly, the basic orientation in the non-locked state of the microscope hanging mount can be set for both configurations of the medical microscope 9 in FIGS. 3 and 4 in accordance with the specified/desired use-observation direction N in three-dimensional space.

The configuration shown in FIG. 5 differs from the configuration in FIG. 3 in that a non-vertical use-observation direction N is set, with the same observation direction 20. In other words, the microscope body 21 with a microscope axis 21A tilted relative to the vertical direction (and a correspondingly aligned use-observation direction N) is to be positioned in the non-locked state of the ball joint system 15 relative to the patient to be examined/treated. To this end, the pivot unit 31 is opened slightly and the center of gravity translation unit 41 moves the microscope body 21 relative to the ball joint system (also in FIG. 5, the base body 19B no longer engages centrally with the center of gravity translation unit 41), so that the desired orientation of the use-observation direction N is achieved without changing the observation direction 20.

FIG. 6 schematically shows a rotation unit that can be used as an interface, the rotation unit comprising two halves 63A, 63B that can rotate relative to each other about a section 10A of the axis of symmetry (arrow 17, see also FIG. 2). Each of the halves comprises a pair of openings 65 for the binocular partial beam paths 61A, 61B. The openings 65 are indicated exemplarily as circular in FIG. 6, but their shape is not limited and they can also be formed oval, angular, or banana-shaped extending around the axis of rotation (with additional apertures in the beam path, if necessary). The openings 65 restrict the acquired light that is assigned to the two partial beam paths 61A, 61B and transmitted by the rotation unit. In a basic setting of the microscope unit, the openings 65 can be aligned with each other, for example, in such a way that there is maximum overlap and, thus, minimum light loss, for example. In the rotation angle shown in FIG. 6, one can see that the pairs of openings 65 are rotated relative to each other, but the overlap of the openings is still large enough to allow sufficient image information to pass through. Depending on the required shift of the field of view (which also depends, inter alia, on the setup and working distance of the microscope), rotation angles in the range of ±20° (or more) or ±10° or ±5° in each direction of rotation can be provided with a tolerable loss of light (within the limits of a field of view restriction that does not limit the operation).

FIG. 7 schematically shows an exemplary optical setup of a pivot unit that can be used as an interface. One can see optical components 71A, 71B (e.g., mirrored prisms/roof prisms) of two deflection prism systems 71 assigned to partial beam paths 61A, 61B (see also FIG. 8 with respect to the course of the beam path 61A in the deflection prism system of the partial beam path 61A). The optical components 71 are held in a housing (not shown) that has a mechanism allowing the indicated rotations (arrows 73). The mechanism allows a deflection of the overall path of the partial beam paths 61A, 61B about a pivot axis 31A (see also FIG. 2). The deflection is accompanied by a pivoting of the microscope unit relative to the ocular optical system about the pivot axis 31A and is shown in FIG. 7 by an arrow 31B and an angled course of the axis of symmetry (sections 10B, 10C), whereby the pivot axis 31A extends orthogonally to the sections 10B, 10C of the axis of symmetry, for example. For example, the two rotations indicated for each of the partial beam paths can be coupled, in particular, performed in opposite directions. The optical components are configured (in as compact a structure as possible) and arranged relative to each other in such a way that, in the desired deflection angle range, the acquired light beam is guided essentially completely through the deflection prism systems 71. Deflection angles in the range of ±20° (or even more, up to several tens of degrees, can be possible), whereby also for the pivot unit, the deflection angle range and the shift of the field of view achievable with it also depend, inter alia, on the setup and working distance of the microscope,. Furthermore, the pivot unit can also have a sequence of such deflection prism systems for greater flexibility in beam guidance.

The use of prism systems (or, in general, mirror-based or prism and mirror combining setups) in the opto-mechanical system, in particular, in the context of the pivot unit, can also allow a reduction or even elimination of imaging errors (such as vignetting or cropping of the light beam) and of double images.

The preceding description illustrates the wide range of possibilities for shifting and orienting the field of view of the microscope, in particular using the disclosed components, the microscope mounting, the opto-mechanical system, the rotary joint 30 and/or the gravity translation unit 41. When using the microscope according to the invention, the result is a high level of ergonomics for the operator of the microscope. The concepts disclosed herein allow the operator to perform in simple manner a (first) positioning of the microscope, e.g., for an ergonomic sitting/standing position that can be maintained in a relaxed manner even during operations lasting several hours. The inventive concept allows that the microscope can be positioned in three-dimensional space without major gravity-induced movement fluctuations.

It is explicitly emphasized that all features disclosed in the description and/or claims are to be considered separate and independent of each other for the purpose of the original disclosure as well as for the purpose of limiting the claimed invention, regardless of the combinations of features in the embodiments and/or claims. It is explicitly stated that all range indications or indications of groups of units disclose any possible intermediate value or subgroup of units for the purpose of the original disclosure as well as for the purpose of limiting the claimed invention, in particular, also as the limit of a range indication.

1. A medical microscope comprising:
a microscope ocular system with a binocular, wherein the binocular provides an observation direction into the microscope,
a microscope body with at least one objective lens for capturing light from a field of view, wherein the microscope body is configured to transmit the light to the microscope ocular system and has a microscope axis extending from the microscope body to the field of view, and
a microscope hanging mount for mounting the microscope to a support system, wherein the microscope hanging mount is configured as part of a ball joint system and, in a non-locked state of the microscope hanging mount, provides rotatability of the medical microscope about a reference point of the microscope hanging mount,
characterized in that
the microscope hanging mount is positioned relative to a center of gravity of the microscope in such a way that, when the microscope is attached to the support system in a non-locked state of the microscope hanging mount, the microscope body assumes a basic orientation in which the microscope axis extends in a predetermined use-observation direction in three-dimensional space and the observation direction extends at a predetermined use-angle range with respect to a hanging mount axis extending through the center of gravity and the microscope hanging mount.

2. The medical microscope of claim 1, wherein the microscope axis extends parallel offset or coaxially to the hanging mount axis or wherein the microscope axis extends at an angle in a range from 0° to 5° to the hanging mount axis or in a range from 20° to 90° with respect to the hanging mount axis.

3. The medical microscope of claim 1, wherein the use-angle range comprises angles in the range from 0° to 90°, in particular in the range from 25° to 70°, with respect to the hanging mount axis.

4. The medical microscope of claim 1, further comprising a center of gravity translation unit, which is arranged between the microscope hanging mount and the microscope body, in particular between the microscope hanging mount and an ocular base body of the microscope ocular system, and is configured for translating the microscope body in one or two directions, in particular for changing a position of the center of gravity relative to the microscope hanging mount.

5. The medical microscope of claim 4, wherein the center of gravity translation unit is configured to position the center of gravity of the medical microscope with regard to adjusting the basic orientation to at least two configurations of the medical microscope, which differ in the position of the center of gravity, so that the basic orientation in the at least two configurations of the medical microscope is settable in accordance with the predetermined use-observation direction in three-dimensional space when the microscope hanging mount is in the non-locked state.

6. The medical microscope of claim 1, wherein the microscope is provided with at least one degree of freedom of movement, which provides an orientability of the microscope axis with respect to the microscope ocular system, and the basic orientation is present in a basic setting of the medical microscope with respect to the degree of freedom of movement, wherein the basic setting provides a deflection in two opposite directions enabled by the degree of freedom.

7. The medical microscope of claim 6, wherein the hanging mount axis of the medical microscope is defined in the basic setting of the medical microscope.

8. The medical microscope of claim 1, wherein the microscope hanging mount is arranged on the microscope ocular system and/or on the microscope body.

9. The medical microscope of claim 1, further comprising
an opto-mechanical system arranged between the microscope ocular system and the microscope body and feeding light captured with the microscope body to the binocular,
wherein the opto-mechanical system is configured for a pivot movement of the microscope body about a pivot axis and comprises a first rotation unit for a rotational movement of the microscope body about a first rotation axis given by the first rotation unit, wherein the first rotation axis extends coaxially, parallel or at an angle in the range from 0° to 5° to a section of the optical axis of symmetry that extends through the first rotation unit.

10. A microscope system comprising:
a support system, which is configured in particular as a floor, wall or ceiling stand or as a permanently mounted support system, and
a medical microscope of claim 1, which is mounted to the support system with a microscope hanging mount of a microscope ocular system of the medical microscope,
wherein
the microscope hanging mount provides, in particular in a non-locked state, multi-axis freedom of movement, so that the mounted medical microscope assumes a basic orientation in the non-locked state, in which a microscope axis of the medical microscope extends in a predetermined use-observation direction in three-dimensional space, in particular in a vertical direction downwards or at an angle of 0° to 5° to the vertical direction, and an observation direction at a predetermined use-angle in the range of 0° to 90°, in particular in the range from 25° to 70°, to a hanging mount axis extending through the center of gravity and the microscope hanging mount.

11. The microscope system of claim 10, wherein the support system comprises a microscope mounting arm, which
engages the microscope hanging mount of the microscope ocular system at an angle in the range from 0° to 20° with respect to a vertical direction, in particular vertically from above, and/or
comprises a rotary joint that enables rotatability of the attached microscope, when the microscope hanging mount is locked, wherein a rotation axis of the rotary joint extends in particular coaxially or parallel offset to the use-observation direction, for example, vertically.

12. The microscope system of claim 10, wherein the support system engages the microscope hanging mount of the microscope ocular system at an angle in the range from 0° to 90° with respect to a vertical direction, in particular, obliquely from above.
